(19) United States
(12) Patent Application Publication
(10) Pub. No.: US 2007/0253909 A1
(43) Pub. Date: Nov. 1, 2007

(54) Aqueous Antiseptic Solution and Compatible Cationic Dye for Staining Skin

(75) Inventors: Angel G. Magallon, El Paso, TX (US); Kyle W. Boone, El Paso, TX (US); James R. Bardwell, El Paso, TX (US)

Correspondence Address: SHOOK, HARDY & BACON LLP;INTELLECTUAL PROPERTY DEPARTMENT, 2555 GRAND BLVD, KANSAS CITY, MO, 64108-2613, US

(73) Assignee: Medi-Flex, Inc., Leawood, KS (US)

(21) Appl. No.: 11/381,047

(22) Filed: May 1, 2006

(51) Int. Cl. A61K 31/655; A61K 31/155; A61K 31/135

(52) U.S. Cl. 424/9.6; 514/634; 514/150; 514/647

(57) ABSTRACT

Aqueous antiseptic solutions and compatible dyes for staining skin and methods for making and using such solutions are provided. More specifically, in one embodiment, the present invention relates to an aqueous antiseptic solution comprising an aqueous solution of chlorhexidine or a salt thereof and a cationic dye in an amount sufficient to stain a patient's skin when the aqueous solution is applied thereon.

### CROSS-REFERENCE TO RELATED APPLICATIONS

This application is related by subject matter to the invention disclosed in U.S. Application No.(not yet assigned) (Attorney Docket Number MDFX.128860), filed on even date herewith, entitled Aqueous Antiseptic Solution and Compatible Anionic Dye for Staining Skin, which is assigned or under obligation of assignment to the same entity as this application, and incorporated in this application by reference.

### STATEMENT REGARDING FEDERALLY SPONSORED RESEARCH OR DEVELOPMENT

Not applicable.

### BACKGROUND

Antisepsis is the destruction or inhibition of microorganisms that exist on living tissue. Antiseptics kill or prevent the growth of the microorganisms. Commonly used antiseptics include iodine, boric acid, and alcohol. Another type of antiseptic used is chlorhexidine including its salts, such as, for example, chlorhexidine gluconate (CHG). CHG is an especially effective antiseptic as it exhibits a strong affinity for binding to skin, a high level of antibacterial activity, and prolonged residual effects. It has been found that CHG is a rapid acting, persistent and superior preoperative skin preparation and kills more bacteria than traditional iodophors or alcohol. Further, CHG exhibits rapid activity against both gram-positive and gram-negative bacteria.

An alcohol, such as isopropanol, is commonly used as a solvent for CHG solutions. An example of an alcohol-based CHG solution is ChloraPrep®, which is a 2% w/v CHG and 70% v/v isopropanol solution available from Medi-Flex, Inc. of Leawood, Kans. Because of their flammable properties, however, such alcohol-based solutions may pose a hazard. As a result, some surgical suites and similar clinical settings may prohibit the use of such solutions. Accordingly, it may be desirable to use an aqueous CHG solution in such settings. An aqueous solution is any solution in which water is the primary dissolving medium or solvent.

Because an aqueous CHG solution is a non-colored or clear liquid, it is difficult for the user to see where the liquid has been applied. However, it is important in many situations of using an antiseptic, such as an aqueous CHG solution, for an individual to know where the antiseptic has been applied. For example, antiseptics are often applied to a patient's skin just prior to surgery. It is essential that an individual, such as a nurse or surgeon, be able to see where the preoperative liquid has been applied. In such cases, if the preoperative liquid were to be colored such that the liquid would stain a patient's skin when applied, it would be easier for an individual to discern not only that the antiseptic has been applied but also where the liquid has been applied to the patient's body.

Although colorants have been added to CHG solutions in some applications, such as handwashes, none of these applications has suggested the addition of a dye in amount sufficient to stain or color a patient's skin. In particular, coloring has been added to CHG solutions to provide only a weak color for aesthetic purposes. Numerous problems are encountered when increased levels of a colorant, such as a tint or dye, are added to aqueous CHG solutions. For example, higher concentrations of dyes are generally incompatible with aqueous CHG solutions. When a dye is added to CHG solutions, the shelf life of the solution may be shortened and/or the colored solution may become unstable. In other words, the addition of a dye may reduce the efficacy of the CHG solution. A further problem is colorant may settle out of the solution, causing a non-uniform distribution of the colored solution when applied.

### BRIEF SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to aqueous antiseptic solutions comprising an aqueous solution of chlorhexidine or a salt thereof and a compatible dye in an amount sufficient to stain a patient's skin. A dye is compatible in accordance with embodiments of the present invention when an amount sufficient to dye a patient's skin may be dissolved in solution with little or no visible precipitant being formed. Accordingly, a compatible dye used herein will provide an ability to stain a patient's skin when the aqueous antiseptic solution is applied without reducing the efficacy of the chlorhexidine or salt thereof.

Accordingly, in one aspect, an embodiment of the present invention is directed to an aqueous antiseptic solution comprising an aqueous solution of chlorhexidine or a salt thereof, and a cationic dye in an amount sufficient to stain a patient's skin when applied.

Another embodiment of the present invention is directed to an aqueous antiseptic solution comprising an aqueous solution of from 2.0% w/v to 6.0% w/v of chlorhexidine or a salt thereof, and from 0.004% w/v to 0.25% w/v of a cationic dye.

In a further aspect of the invention, an embodiment is directed to a method for preparing an aqueous antiseptic solution having a compatible dye. The method includes adding an amount of cationic dye sufficient to stain a patient's skin to an aqueous solution of chlorhexidine or a salt thereof.

Yet another embodiment of the present invention is directed to a method of providing an aqueous antiseptic solution and a compatible dye. The method includes providing an aqueous solution of chlorhexidine or a salt thereof. The method also includes providing a cationic dye, wherein the cationic dye is provided in an amount that when combined with the aqueous solution of chlorhexidine or the salt thereof an antiseptic solution is provided that is capable of staining a patient's skin when applied.

In another aspect of the present invention, an embodiment is directed to an aqueous antiseptic solution comprising an aqueous solution of chlorhexidine or a salt thereof, an anionic dye in an amount sufficient to stain a patient's skin when applied, and a cationic excipient.

A further embodiment of the present invention is directed to an aqueous antiseptic solution comprising an aqueous solution of from 2.0% w/v to 6.0% w/v of chlorhexidine or a salt thereof, from 0.07% w/v to 0.15% w/v of an anionic dye, and a cationic excipient, wherein the minimum molar ratio of the cationic excipient to the anionic dye is based on the charge ratio between the cationic excipient and the anionic dye.

In another embodiment, an aspect of the invention is direct to a method for preparing an aqueous antiseptic solution having a compatible dye. The method includes adding to an aqueous solution of chlorhexidine or a salt thereof: an amount of anionic dye sufficient to stain a patient's skin, and a cationic excipient.

Still further, an embodiment of the present invention is directed to a method of providing an aqueous antiseptic solution and a compatible dye. The method includes providing an aqueous solution of chlorhexidine or a salt thereof. The method also includes providing an anionic dye, wherein the anionic dye is provided in an amount that when combined with the aqueous solution of chlorhexidine or the salt thereof an antiseptic solution is provided that is capable of staining a patient's skin when applied. The method further includes providing a cationic excipient.

Additional aspects of the invention, together with the advantages and novel features appurtenant thereto, will be set forth in part in the description that follows, and in part will become apparent to those skilled in the art upon examination of the following, or may be learned from the practice of the invention. The objects and advantages of the invention may be realized and attained by means, instrumentalities, and combinations particular pointed out in the appended claims.

### DETAILED DESCRIPTION

Embodiments of the present invention are directed to aqueous antiseptic solutions having chlorhexidine or a salt thereof as the antiseptic and a compatible dye in an amount sufficient to visibly stain or otherwise color a patient's skin. Antiseptic solutions of the present invention exhibit excellent antimicrobial activity in an aqueous solution, thereby eliminating hazards associated with alcohol-based solutions. Additionally, the inclusion of a compatible dye provides substantial benefit by allowing users to readily identify where the antiseptic solutions have been applied to a patient.

As used herein the term “aqueous solution” is used to refer to a solution in which water is the primary dissolving medium or solvent. In other words, the term “aqueous solution” refers to a solution in which water is the solvent in the largest concentration by volume.

Antiseptics that may be employed to provide antimicrobial activity for compositions of the present invention include chlorhexidine and salts thereof. The chlorhexidine salt used in preferred embodiments is CHG. Although CHG is the chlorhexidine salt presented in the examples below, the present invention should not be limited to CHG as other types of chlorhexidine salts are suitable. Examples of such suitable chlorhexidine salts include gluconate, acetate, chloride, bromide, nitrate, sulphate, carbonate, and phosphanilate. The concentration of chlorhexidine or a chlorhexidine salt in the aqueous antiseptic solution may vary within various embodiments of the present invention. However, in preferred embodiments, the concentration of chlorhexidine or a chlorhexidine salt is about 2.0% w/v to about 6.0% w/v. Preferably, the aqueous antiseptic solution includes about 2.0% w/v CHG.

In some embodiments of the present invention, the aqueous antiseptic solution comprises an aqueous solution of chlorhexidine or a salt thereof, an anionic dye in an amount sufficient to visibly stain a patient's skin when applied, and a cationic excipient in an amount sufficient to prevent the anionic dye from forming a precipitant with the chlorhexidine or salt thereof. Anionic dyes, including FD&C dyes, form a precipitant with chlorhexidine, even at very low concentrations. As such, adding an anionic dye alone to an aqueous chlorhexidine solution removes a significant fraction of the chlorhexidine from solution, decreasing the efficacy of the solution. The precipitant is believed to be the insoluble salt of a chlorhexidine cation and at least one dye anion. The solubility product of the chlorhexidine dye salt, assuming one anion, was measured to be less than 10⁻⁹ for all such anionic dyes. However, if the negative charge of an anionic dye is “hidden” from the chlorhexidine by a cationic excipient, the chlorhexidine-dye salt will not immediately form. The cationic excipient makes a reversible association with the anionic dye to protect its structure. The chlorhexidine-dye salt association, however, is an irreversible association when its solubility product is exceeded. While the excipient-dye association may be kinetically favored, the chlorhexidine-dye complex is thermodynamically favored because the reverse reaction rate is practically zero. However, in preferred embodiments, the addition of a cationic excipient will prevent a chlorhexidine-dye salt for the expected usage life of about 72 hours.

The interaction between anionic dyes and cationic excipients in aqueous antiseptic solutions of the present invention comprises a reversible association of the anionic dyes with cationic excipient micelles by ionic interactions. Micellation refers to a process in which submicroscopic molecules aggregate, as a droplet in a colloidal system. The driving force for aggregation is the gain in entropy, according to Boltzmann's principle, of the water molecules formerly associated with the hydrophobic molecules and now associated with other water molecules. Entropy is increased because water has more allowable states next to polarized molecules (hydrophilic) than next to non-polarized molecules (hydrophobic). The increase in ionic strength associated with dissolving an anionic dye packs micellular cationic excipient molecules even closer together, increasing the density of positive charge at the surface of the micelle.

Anionic dyes that may be employed within aqueous antiseptic solutions of the present invention include FD&C dyes, such as, for example, FD&C Blue No. 1 (Brilliant Blue FCF), FD&C Blue No. 2 (Indigo Carmine), FD&C Green No. 3 (Fast Green FCF), FD&C Red No. 3 (Erythrosine), FD&C Red No. 40 (Allura Red), FD&C Yellow No. 5 (Tartrazine), and FD&C Yellow No. 6 (Sunset Yellow FCF). In embodiments, the concentration of anionic dye sufficient to stain a patient's skin but otherwise compatible with chlorhexidine via the addition of a cationic excipient may range from about 0.07% w/v to about 0.15% w/v. In preferred embodiments, the concentration of anionic dye is about 0.13% w/v.

Certain types of cationic excipient may be employed with the scope of the present invention to provide a compatible chlorhexidine-anionic dye solution. In some embodiments, the cationic excipient comprises a cationic detergent. In some embodiments, a cationic excipient containing quaternary nitrogen may be used. Such cationic excipients may include, for example, cetylpyridinium chloride (CPC), hexadecyl trimethyl ammonium bromide, benzethonium chloride, and benzalkonium chloride. The concentration of cationic excipient is dependent upon the charge ratio between the dye and cationic excipient used to prepare the aqueous antiseptic solution. For example, a cationic excipient having a single positive charge and an anionic dye having two negative charges would result in a molar ratio of cationic excipient to anionic dye of about 2 to 1.

In other embodiments of the present invention, the aqueous antiseptic solution comprises an aqueous solution of chlorhexidine or a salt thereof and a cationic dye in an amount sufficient to visibly stain a patient's skin when applied. Non-limiting examples of such cationic dyes include crystal violet, acriflavine, bismarck brown, malachite green, methyl green, Victoria pure blue BO, and azure C. In contrast to anionic dyes, cationic dyes were found to be compatible with aqueous chlorhexidine solutions without the addition of a cationic excipient. However, some cationic dyes include a chloride ion or other ion that forms a precipitant with chlorhexidine as the concentration is increased. For example, the compatibility of cationic dyes having a chloride ion with aqueous chlorhexidine solutions decreases after the solubility product of chlorhexidine and chloride is exceeded at about 0.05% w/v dye. As such, in embodiments, the concentration of cationic dye sufficient to stain a patient's skin but otherwise compatible with chlorhexidine may range from about 0.004% w/v to about 0.25% w/v. In preferred embodiments, the concentration of cationic dye is about 0.05% w/v.

Aqueous antiseptic solutions in accordance with some embodiments of the present invention may employ additional components. For example, in some embodiments, the aqueous antiseptic solution may employ a surfactant. Examples of such suitable surfactants include polyvinyl pyrrolidone (PVP) (average molecular weight 10,000) and PVP (average molecular weight 1,300,000). In embodiments, the concentration of surfactant in an aqueous antiseptic solution may generally range from about 0.5% w/v to about 5% w/v. In a preferred embodiment, PVP (average molecular weight 10,000) in added as a surfactant in a concentration of about 1% w/v.

Additionally, in some embodiments, aqueous antiseptic solutions may employ a solubilization aid. Examples of such suitable solubilization aids include polyethylene glycol (PEG) (average molecular weight 200), PEG (average molecular weight 400), and glycerol. The concentration of a solubilization aid in an aqueous antiseptic solution of embodiments of the present invention may generally range from about 1% v/v to about 49% v/v. In a preferred embodiment, PEG (average molecular weight 200) is added as a solubilization aid in a concentration of about 1% v/v to about 49% v/v.

Additional additives may also be employed within aqueous antiseptic solutions of further embodiments of the present invention, including, for example, small concentrations of alcohol. Such additives would be employed in acceptable manners and amounts established in the art.

In some embodiments, an aqueous antiseptic solution and compatible dye may be provided in conjunction with a liquid applicator. For example, a liquid applicator may be provided that comprises a hollow body defining an internal chamber to receive at least one ampoule formed of a frangible material. In some embodiments, the ampoule(s) contain an aqueous antiseptic solution having a dye therein as described hereinabove. The ampoule(s) may be fractured, and the colored aqueous antiseptic solution may be applied to the desired surface. In other embodiments, the ampoule(s) contain an untinted aqueous chlorhexidine solution, and the liquid applicator includes a porous element with a compatible dye therein. The porous element is positioned such that upon fracturing the ampoule(s), the untinted aqueous antiseptic solution is passed through the porous element and dye is transferred to the solution, which may then be applied to the desired surface. Examples of such liquid applicators are further described in: U.S. Pat. No. 6,729,786; 6,991,393, and U.S. patent application Ser. No. 11/254,318, filed Oct. 20, 2005; each of which is herein incorporated by reference in its entirety.

The ampoule(s) may be numerous different shapes and sizes depending on the amount of liquid needed to be applied. For example, a liquid applicator may include long cylindrical ampoule(s) or may contain vial-type ampoule(s). Furthermore, more than one ampoule may be received by the body. Preferably, the ampoule(s) are formed of glass, although other materials are entirely within the scope of the present invention. The wall of the ampoules is of a thickness sufficient to contain the desired liquid during transport and storage, yet allow the ampoule to be fractured upon the application of localized pressure.

The body of the liquid applicator may take many forms. The body has an internal chamber that is adapted to receive at least one ampoule. The body may also be shaped to hold multiple ampoules. In one form, the body is shaped to generally conform to the ampoule(s) contained within the body.

The porous element of the present invention also may take many forms. The porous element may be a porous plug and/or a porous pad. In other words, a porous plug may be located within the body of the applicator between the ampoule and an open end of the body. Additionally or alternatively, a porous pad may be located at an open end of the body. In some embodiments, a compatible dye (e.g., a cationic dye or an anionic dye/cationic excipient composition) may be provided in and/or on the porous element. The porous element is positioned such that when the ampoule(s) is fractured, the untinted aqueous antiseptic solution flows through the porous element and dye is transferred to the solution to be applied. The porous element may be made of any porous material that allows liquid to flow through the material. The porous element may be, but is not limited to, a fabric, foam or a felt material. Dye may be saturated throughout the porous element or may be placed only on part of the element.

The ampoule(s) contained within the body of the applicator may be broken by any method known to those skilled in the art. These include, but are not limited to, squeezing the walls of the body inwardly to break the ampoule(s), using a lever or other mechanism to break the ampoule(s), or utilizing projecting wings with tappets.

### EXAMPLES

Embodiments of the present invention will now be further illustrated by the following, non-limiting examples.

### Example 1

The compatibility of an anionic dye alone with an aqueous CHG solution (i.e., without the addition of a cationic excipient) was tested. A 0.13% w/v anionic dye solution was prepared by dissolving 0.13 g FD&C Yellow 6 in 100 ml of distilled water. A 20% w/v aqueous CHG solution was then added drop wise to the dye solution. After two drops of the aqueous CHG solution were added to the dye solution, precipitant was formed, demonstrating the incompatibility of the dye alone with the aqueous CHG solution.

### Example 2

To test the compatibility of anionic dyes and cationic excipients, a number of solutions were prepared with different anionic dyes and cationic excipients. The cationic excipients tested included: CPC, hexadecyl trimethyl ammonium bromide, benzethonium chloride, and benzalkonium chloride. The anionic dyes tested included: FD&C Green No. 3 (Fast Green FCF), FD&C Yellow No. 5 (Tartrazine), FD&C Red No. 40 (Allura Red), FD&C Yellow No. 6 (Sunset Yellow FCF), FD&C Blue No. 1 (Brilliant Blue FCF), FD&C Blue No. 2 (Indigo Carmine), and FD&C Red No. 3 (Erythrosine). The chemical structure and chemical category of each of these dyes are presented below.

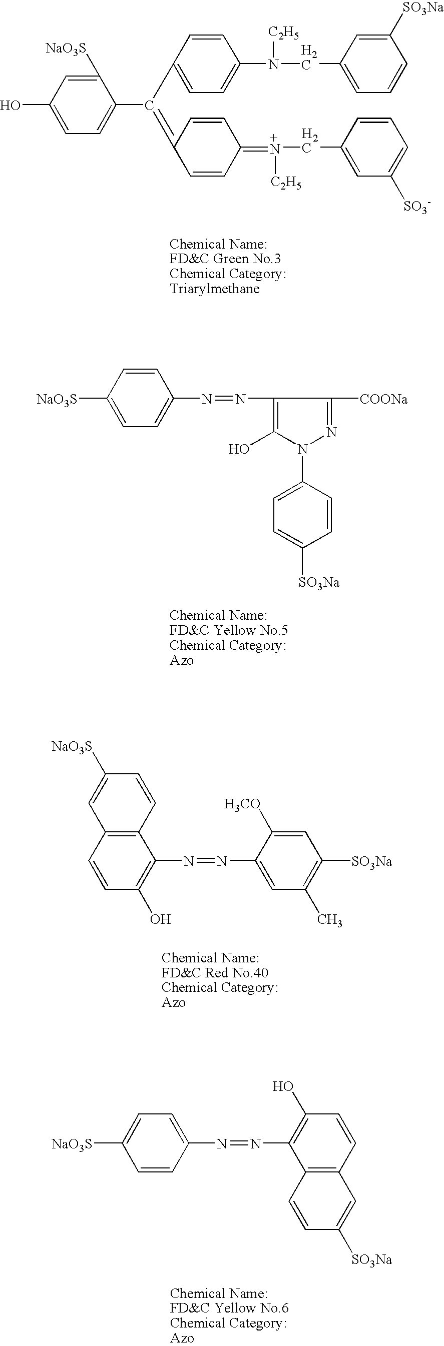

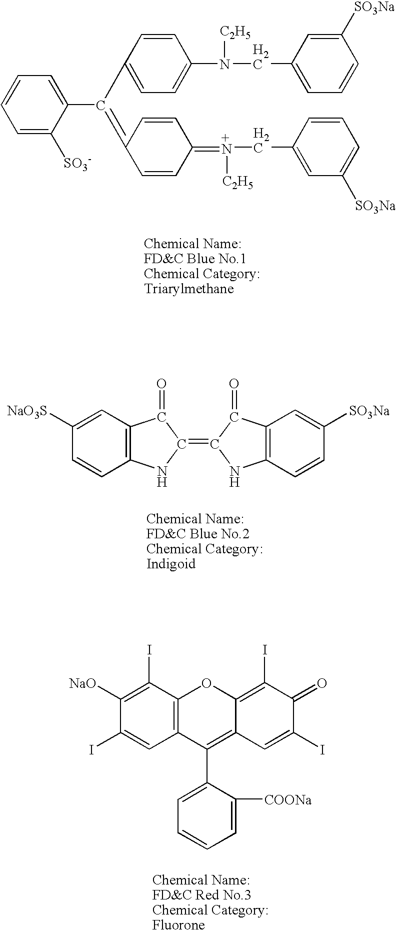

The compatibility of anionic dyes and cationic excipients were tested as follows. First, 0.1 grams of each anionic dye were placed in separate 40-ml beakers. 20 ml of 4 mM excipient solution were added to each 40-ml beaker. Each of the cationic excipients solubilized the anionic dyes.

### Example 3

A titration experiment was designed to determine the appropriate cationic excipient to anionic dye molar ratio. The experiment was performed using CPC as the cationic excipient and FD&C Yellow No. 6 as the dye. The titration was done by placing a known volume of 4mM CPC solution in a beaker and titrating with a 2% w/v solution of FD&C Yellow No. 6. The solution containing CPC and FD&C Yellow No. 6 was added drop wise to an aqueous 2.0% w/v CHG solution. Results indicated that the minimum molar ratio of CPC to FD&C Yellow No. 6 was approximately 2 to 1. This result represents the charge ratio between the two components.

### Example 4

An aqueous 2.0% w/v CHG solution having an anionic dye was formulated using a Class A 100-ml volumetric flask. The procedure included dissolving 0.30 grams CPC and 0.13 grams FD&C Yellow No. 6 with 6.0 ml of 50/50% v/v of isopropanol and distilled water. Separately, 1.0 grams of PVP (average molecular weight 10,000) was completely dissolved in 30.0 ml of distilled water. Once dissolved, the PVP solution was incorporated with the dye/excipient solution. Next, 5 ml of PEG (average molecular weight 200) was added. Additionally, 10.6 grams of 20% w/v CHG solution was added. Finally, distilled water was added to the flask until the 100-ml mark was reached.

### Example 5

A tinted aqueous 6.0% w/v CHG solution using an anionic dye and cationic excipient was prepared using a Class A 100-ml volumetric flask. First, 0.30 grams CPC and 0.13 grams FD&C Yellow No. 6 was dissolved in the flask using 6.0 ml of 50/50% v/v of isopropanol and distilled water. Next, 31.8 grams of 20% w/v CHG solution was added. Distilled water was then added to the flask until the 100-ml mark was reached.

### Example 6

In this example, a liquid applicator was prepared having an untinted aqueous CHG solution in an ampoule and anionic dye/cationic excipient composition contained in a porous element. To prepare the aqueous CHG solution, 1 gram of PVP (average molecular weight 10,000) was dissolved in 30 ml of distilled water. Then, 5 ml of PEG (average molecular weight 200) was added. Additionally, 10.6 grams of 20% w/v aqueous CHG solution was provided and dissolved water was added until the 100-ml mark was reached. The aqueous CHG solution was added to a glass ampoule, which was then sealed and placed inside the hollow body of the liquid applicator.

A porous element having an anionic dye/cationic excipient composition was prepared for the liquid applicator as follows. First, 100 ml of a dye solution was prepared by adding 2.0 grams of FD&C Yellow No. 6 and 4.6 grams of CPC in 100 ml of 50/50% v/v of isopropanol and distilled water. The porous element was dipped in the dye solution for 1 minute and then air-dried for 24 hours. The porous element was then secured to the end of the applicator body.

Upon fracturing of the ampoule, the untinted aqueous CHG solution flows through the porous element containing the anionic dye/cationic excipient composition. Dye and cationic excipient are thereby transferred to the aqueous CHG solution as it flows through the porous element. The resulting colored aqueous CHG solution may be applied to a desired surface, such as a patient's skin, thereby both disinfecting and visibly staining the surface.

### Example 7

To prove that chlorhexidine will not precipitate with cationic ammonium-containing dyes, the following dyes were tested: crystal violet, Victoria pure blue BO, methyl green, malachite green, acriflavine, and bismarck brown. The chemical structure and chemical category of each of these dyes are presented below. Crystal violet, malachite green, and Victoria pure blue BO all belong to the same chemical family, triarylmethane. Bismark brown and acriflavine each belong to a different chemical family, azo and acridine, respectively.

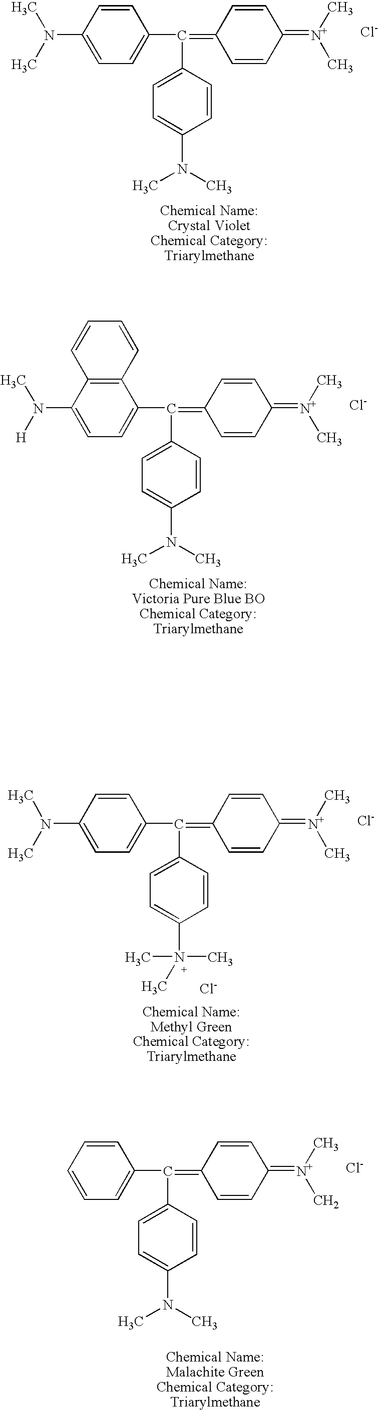

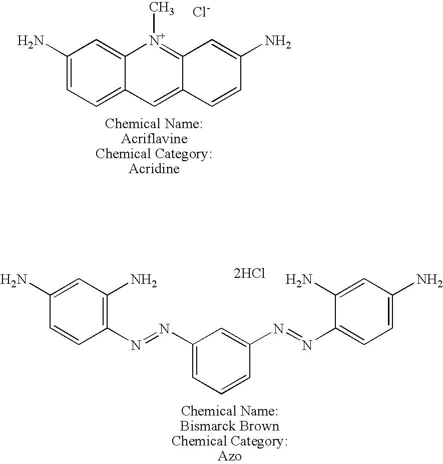

Aqueous solutions of 2.0% w/v CHG and 0.05% w/v dye were prepared for each of the dyes indicated above to test the stability of the solutions. Each aqueous solution was prepared using a Class A 100 ml volumetric flask, in which 0.050 grams of dye were dissolved in 30.0 ml of distilled water. Additionally, 10.6 g of 20% w/v aqueous CHG solution was added to the flask. Distilled water was then added to the flask until the 100-ml mark was reached. The solutions were stored for three months at room temperatures. No visible precipitant formed in any of the solutions, indicating that the solutions were stable and the dyes were compatible for at least three months at a concentration of 0.05% w/v.

### Example 8

The compatibility of cationic ammonium-containing dyes with aqueous CHG solutions suggested that compatibility may not be limited to ammonium-containing dyes and that any cationic dye may be compatible. Accordingly, an aqueous CHG solution with a cationic dye that does not contain an ammonium group was prepared to test its compatibility. In particular, the dye tested was azure C, which is a cationic dye that belongs to the thiazin family. Its positive charge comes from a tertiary sulfur atom instead of a quaternary nitrogen atom. The chemical structure and chemical category of azure C is presented below.

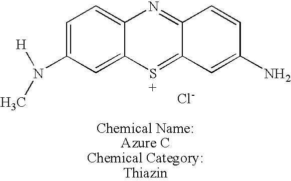

An aqueous solution of 2.0% w/v CHG and 0.05% w/v azure C dye was prepared to test the stability of the solution. The solution was prepared similar to the preparation of the solutions in Example 7. First, 0.050 grams of azure C dye were dissolved in 30.0 ml of distilled water using a Class A 100 ml volumetric flask. Additionally, 10.6 g of 20% w/v aqueous CHG solution was added to the flask. Distilled water was then added to the flask until the 100-ml mark was reached. The solutions were stored for three months at room temperatures. The solution showed no visible precipitant, indicating that the solution was stable and the dye was compatible for at least three months at a concentration of 0.05% w/v.

### Example 9

An aqueous 6.0% w/v CHG solution having a cationic dye was prepared using a Class A 100-ml volumetric flask. First, 0.050 grams of crystal violet was dissolved in 30.0 ml of distilled water. Next, 31.8 grams of 20% w/v CHG solution was added. Distilled water was then added to the flask until the 100-ml mark was reached.

### Example 10

In this example, a liquid applicator was prepared having an untinted aqueous CHG solution in an ampoule and cationic dye contained in a porous element. To prepare the aqueous CHG solution, 10.6 grams of 20% w/v aqueous CHG solution was provided and dissolved water was added until the 100-ml mark was reached. The aqueous CHG solution was added to a glass ampoule, which was then sealed and placed inside the hollow body of the liquid applicator.

A porous element having a cationic dye was prepared for the liquid applicator as follows. First, 100 ml of a dye solution was prepared by adding 0.3 grams of crystal violet dye in 100 ml of 50/50% v/v of isopropanol and distilled water. The porous element was dipped in the dye solution for 1 minute and then air-dried for 24 hours. The porous element was then secured to the end of the applicator body.

Upon fracturing of the ampoule, the untinted aqueous CHG solution flows through the porous element containing the cationic dye. Dye is thereby transferred to the aqueous CHG solution as it flows through the porous element. The resulting colored aqueous CHG solution may be applied to a desired surface, such as a patient's skin, thereby both disinfecting and visibly staining the surface.

As can be understood, the present invention provides an aqueous antiseptic solution comprising an aqueous solution of chlorhexidine or a salt thereof and a compatible dye in an amount sufficient to stain a patient's skin.

The present invention has been described in relation to particular embodiments, which are intended in all respects to be illustrative rather than restrictive. Since many possible embodiments may be made of the invention without departing from the scope thereof, it is to be understood that all matter herein set forth or shown in the accompanying drawings is to be interpreted as illustrative and not in a limiting sense. Alternative embodiments will become apparent to those of ordinary skill in the art to which the present invention pertains without departing from its scope. For example, although embodiments of the present invention have been described with respect to disinfecting and coloring a patient's skin, in further embodiments, the aqueous antiseptic solution may be used to disinfect and color other materials and surfaces, such as medical equipment, for example.

From the foregoing, it will be seen that this invention is one well adapted to attain all the ends and objects set forth above, together with other advantages which are obvious and inherent to the system and method. It will be understood that certain features and subcombinations are of utility and may be employed without reference to other features and subcombinations. This is contemplated and within the scope of the claims.

What is claimed is:

1. An aqueous antiseptic solution comprising an aqueous solution of chlorhexidine or a salt thereof, and a cationic dye in an amount sufficient to stain a patient's skin when applied.

2. The aqueous antiseptic solution of claim 1, wherein the salt of chlorhexidine comprises at least one of gluconate, acetate, chloride, bromide, nitrate, sulphate, carbonate, and phosphanilate.

3. The aqueous antiseptic solution of claim 1, wherein the concentration of the chlorhexidine or the salt thereof is from 2.0% w/v to 6.0% w/v.

4. The aqueous antiseptic solution of claim 1, wherein the concentration of the chlorhexidine or the salt thereof is 2.0% w/v.

5. The aqueous antiseptic solution of claim 1, wherein the cationic dye comprises at least one of crystal violet, acriflavine, bismarck brown, malachite green, methyl green, Victoria pure blue BO, and azure C.

6. The aqueous antiseptic solution of claim 1, wherein the concentration of the cationic dye is from 0.004% w/v to 0.25% w/v.

7. The aqueous antiseptic solution of claim 1, wherein the aqueous antiseptic solution further comprises a surfactant.

8. The aqueous antiseptic solution of claim 7, wherein the surfactant is polyvinyl pyrrolidone

9. The aqueous antiseptic solution of claim 7, wherein the concentration of the surfactant is from 0.5% w/v to 5.0% w/v.

10. The aqueous antiseptic solution of claim 1, wherein the aqueous antiseptic solution further comprises an solubilization aid.

11. The aqueous antiseptic solution of claim 10, wherein the solubilization aid comprises at least one of polyethylene glycol and glycerol.

12. The aqueous antiseptic solution of claim 10, wherein the concentration of the solubilization aid is from 1.0% v/v to 49% v/v.

13. A method of disinfecting a patient's skin comprising applying the aqueous antiseptic solution of claim 1 to the patient's skin.

14. An aqueous antiseptic solution comprising an aqueous solution of from 2.0% w/v to 6.0% w/v of chlorhexidine or a salt thereof, and from 0.004% w/v to 0.25% w/v of a cationic dye.

15. The aqueous antiseptic solution of claim 14, wherein the salt of chlorhexidine comprises at least one of gluconate, acetate, chloride, bromide, nitrate, sulphate, carbonate, and phosphanilate.

16. The aqueous antiseptic solution of claim 14, wherein the cationic dye comprises at least one of crystal violet, acriflavine, bismark brown, malachite green, methyl green, Victoria pure blue BO, and azure C.

17. The aqueous antiseptic solution of claim 14, wherein the aqueous antiseptic solution further comprises a surfactant.

18. The aqueous antiseptic solution of claim 14, wherein the aqueous antiseptic solution further comprises an solubilization aid.

19. A method for preparing an aqueous antiseptic solution having a compatible dye, the method comprising adding an amount of cationic dye sufficient to stain a patient's skin to an aqueous solution of chlorhexidine or a salt thereof.

20. The method of claim 19, wherein the concentration of the chlorhexidine or the salt thereof is from 2.0% w/v to 6.0% w/v.

21. The method of claim 19, wherein the concentration of the cationic dye is from 0.004% w/v to 0.25% w/v.

22. A method of providing an aqueous antiseptic solution and a compatible dye, the method comprising:
providing an aqueous solution of chlorhexidine or a salt thereof; and
providing a cationic dye, wherein the cationic dye is provided in an amount that when combined with the aqueous solution of chlorhexidine or the salt thereof an antiseptic solution is provided that is capable of staining a patient's skin when applied.

23. The method of claim 22, wherein the concentration of the chlorhexidine or the salt thereof is from 2.0% w/v to 6.0% w/v.

24. The method of claim 22, wherein the aqueous solution of chlorhexidine or a salt thereof is provided in at least one ampoule and the cationic dye is provided in at least one porous element of a liquid applicator, wherein the at least one porous element is positioned such that the aqueous solution of chlorhexidine or a salt thereof flows through the at least one porous element when the ampoule is fractured and cationic dye is transferred to the aqueous solution.
